(19) United States
(12) Patent Application Publication
(10) Pub. No.: US 2025/0061644 A1
(43) Pub. Date: Feb. 20, 2025

(54) DATA PROCESSING APPARATUS FOR PROCESSING ORAL MODEL AND OPERATING METHOD THEREFOR

(71) Applicant: MEDIT CORP., Seoul (KR)

(72) Inventors: Sang Hoon KIM, Seoul (KR); Sung Hoon LEE, Seoul (KR)

(73) Assignee: MEDIT CORP., Seoul (KR)

(21) Appl. No.: 18/576,978

(22) PCT Filed: Jul. 5, 2022
(86) PCT No.: PCT/KR2022/009667

§ 371 Date: Aug. 7, 2024

(30) Foreign Application Priority Data
Jul. 6, 2021 (KR) 10-2021-0088574
May 23, 2022 (KR) 10-2022-0063046

(51) Int. Cl. G06T 15/30; G06T 15/40; G06T 19/20

(52) U.S. Cl. CPC G06T 15/30; G06T 15/40; G06T 19/20; G06T 2210/12; G06T 2210/41; G06T 2210/62; G06T 2219/2016

(57) ABSTRACT

A data processing apparatus for processing an intraoral model and a method of operating the data processing apparatus are disclosed. The data processing apparatus includes a memory storing one or more instructions, and a processor configured to execute the one or more instructions to, display an intraoral model including one or more teeth, select a target tooth from among the one or more teeth included in the intraoral model, and display transparently an area of the intraoral model included in a transparent processing area located in a screen direction based on a clipping plane located at a predetermined distance from the target tooth in the screen direction.

### TECHNICAL FIELD

The disclosed embodiment relates to a data processing apparatus for processing an intraoral model and a method of operating the data processing apparatus. Specifically, the disclosed embodiment relates to a data processing apparatus for processing an intraoral model, which processes a partial region of the intraoral model to be transparent, and a method of operating the data processing apparatus.

### BACKGROUND ART

In order to design a dental restoration using computer-aided design (CAD)/CAM (computer-aided manufacturing), it is important to properly set a margin line of a tooth (hereinafter, referred to as a target tooth) requiring restoration. The margin line refers to a boundary point that comes into contact with the target tooth when a restoration is restored. Because the boundary of the restoration is determined according to the setting of the margin line, setting the margin line on the target tooth is important.

However, in a three-dimensional intraoral model, the target tooth and an adjacent tooth are usually displayed together, but a portion of the target tooth may be hidden by the adjacent tooth, which may interfere with setting the margin line.

### DESCRIPTION OF EMBODIMENTS

### Technical Problems

The disclosed embodiments provide a data processing apparatus for processing an intraoral model that can facilitate setting a margin line of a target tooth in an intraoral model, and a method of operating the data processing apparatus.

### Solution to Problem

According to an aspect of the disclosed embodiment, a data processing apparatus includes a memory storing one or more instructions, and a processor configured to execute the one or more instructions to: display an intraoral model including one or more teeth, select a target tooth from among the one or more teeth included in the intraoral model, and display transparently an area of the intraoral model included in a transparent processing area located in a screen direction based on a clipping plane located at a predetermined distance from the target tooth in the screen direction. In this way, by providing a transparent processing area at a predetermined distance from the target tooth, an adjacent tooth adjacent to the target tooth may be processed to be transparent. By processing the adjacent tooth to be transparent, the boundary between the target tooth and a gingival area surrounding the target tooth may be more clearly seen. Therefore, it is possible to easily identify and adjust the margin line of the target tooth.

According to an embodiment, the processor, by executing the one or more instructions, may provide a margin line displayed on the target tooth such that a position of the margin line is adjustable, and adjust and display the position of the margin line according to a user input for adjusting the position of the margin line. An adjacent tooth adjacent to the target tooth may be included in the transparent processing area. As the adjacent tooth included in the transparent processing area are processed to be transparent, a margin line in an interdental area between the target tooth and the adjacent tooth may also be displayed, making it easier for a user to adjust the position of the margin line.

According to an embodiment, the processor, by executing one or more instructions, may provide one or more control points to the margin line and adjust the position of the margin line according to a user input for moving the positions of the one or more control points. The predetermined distance may include a distance from the center point of a bounding box surrounding the target tooth. The predetermined distance may include an average value of horizontal and vertical lengths of a plane perpendicular to an occlusal direction in the bounding box or a value obtained by adding a predetermined offset to a median value of a distance from a center point of the target tooth to a center point of an adjacent tooth.

According to an embodiment, the processor, by executing one or more instructions, may display transparently an area of the intraoral model included in the transparent processing area when a size of the target tooth displayed on a display is greater than or equal to a threshold, and display the intraoral model without setting the transparent processing area when the size of the target tooth displayed on the display is less than the threshold.

According to an embodiment, the processor, by executing one or more instructions, may display a predetermined icon and display the intraoral model without setting the transparent processing area by removing the clipping plane according to an input for selecting the icon.

According to an embodiment, the processor, by executing one or more instructions, may rotate and display the intraoral model in response to a user input for rotating the intraoral model, generate a new transparent processing area based on a new clipping plane by generating a new clipping plane at the predetermined distance in the screen direction from the target tooth rotated according to the rotation of the intraoral model, and display transparently the area of the intraoral model included in the new transparent processing area.

According to an embodiment, the processor, by executing one or more instructions, may detect whether an angle formed between an occlusal direction vector of the target tooth rotated in response to a user input for rotating the intraoral model and a vector in an opposite direction to a screen normal vector is less than a threshold angle, and remove the area processed to be transparent by the transparent processing area by removing the clipping plane based on the angle being detected to be less than the threshold angle.

According to an embodiment, the processor, by executing one or more instructions, may exclude at least a portion of the target tooth from displaying transparently when the at least a portion of the target tooth is located in the transparent processing area according to rotational movement of the target tooth in response to a user input for rotating the intraoral model.

According to another aspect of the disclosed embodiment, a method of operating a data processing apparatus includes displaying an intraoral model including one or more teeth, selecting a target tooth from among the one or more teeth included in the intraoral model, and displaying transparently an area of the intraoral model included in a transparent processing area located in a screen direction based on a clipping plane located at a predetermined distance from the target tooth in the screen direction.

According to another aspect of the disclosed embodiment, a computer-readable recording medium includes one or more instructions for performing a method of processing an intraoral model, wherein the method of processing the intraoral model includes displaying an intraoral model including one or more teeth, selecting a target tooth from among the one or more teeth included in the intraoral model, and displaying transparently the area of the intraoral model included in a transparent processing area located in a screen direction based on a clipping plane located at a predetermined distance from the target tooth in the screen direction.

### Advantageous Effects of Disclosure

According to the method and apparatus for processing a three-dimensional intraoral model, according to the disclosed embodiment, when setting a margin line on a target tooth in a three-dimensional intraoral model, teeth in a certain area based on the target tooth are processed and displayed to be transparent so that a user may set the margin line more accurately and easily.

### BRIEF DESCRIPTION OF DRAWINGS

The present disclosure may be readily understood with a combination of the following detailed descriptions and the accompanying drawings, wherein reference numbers refer to structural elements.

FIG. 1 is a reference diagram for explaining a method of setting a margin line in a three-dimensional intraoral model, according to related technology.

FIG. 2 is a diagram for explaining a digital intraoral model processing system according to an embodiment.

FIG. 3 is a block diagram illustrating a data processing apparatus according to an embodiment.

FIG. 4 is a flowchart illustrating a method of processing a three-dimensional intraoral model in a data processing apparatus according to an embodiment.

FIG. 5 illustrates an example of a three-dimensional intraoral model obtained by a data processing apparatus according to an embodiment.

FIG. 6 is a diagram illustrating a margin line displayed on a target tooth, according to an example.

FIG. 7 is a reference diagram for explaining a method of finding the center point of a target tooth, according to an embodiment.

FIG. 8 is a reference diagram for explaining an example of a method of generating a clipping plane, according to an embodiment.

FIG. 9 is a reference diagram for explaining a method of determining a predetermined distance offset from a center point of a target tooth to generate a clipping plane, according to an embodiment.

FIG. 10 is a diagram showing that an intraoral model area located in a transparent processing area set based on a clipping plane is displayed to be transparent, according to an embodiment.

FIG. 11 is a diagram illustrating an example in which an area displayed to be transparent changes as the position of a three-dimensional intraoral model moves, according to an embodiment.

FIG. 12 shows a target tooth cut along a margin line, according to an example.

FIG. 13 is a reference diagram for explaining a processing method when a portion of a target tooth is included in a transparent processing area, according to an embodiment.

FIG. 14 is a reference diagram for explaining how a transparent processing area is toggled between a displayed state and a non-displayed state according to a user input, according to an embodiment.

### MODE OF DISCLOSURE

This specification clarifies the scope of the inventive concept, explains the principles of the inventive concept, and discloses embodiments so that those of ordinary skill in the art to which the inventive concept pertains may practice the inventive concept. The disclosed embodiments may be implemented in various forms.

Like reference numerals refer to like elements throughout the specification. This specification does not describe all elements of the embodiments, and general content in the technical field to which the inventive concept pertains or content that overlaps among the embodiments is omitted. As used herein, the term “part” or “portion” may be implemented in software or hardware, and according to embodiments, a plurality of “units” may be implemented as one unit or element, or one “unit” may include a plurality of elements. Hereinafter, the working principle and embodiments of the inventive concept will be described with reference to the accompanying drawings.

In the present specification, the image may include at least one tooth or an image representing an oral cavity including at least one tooth (hereinafter, “intraoral image”).

Also, in the present specification, an image may be a two-dimensional image of an object or a three-dimensional model or three-dimensional image representing the object three-dimensionally. Also, in the present specification, an image may refer to data necessary to represent an object in two or three dimensions, for example, raw data obtained from at least one image sensor. In particular, raw data is data obtained to generate an intraoral image and may be data (e.g., two-dimensional data) obtained from at least one image sensor included in an intraoral scanner when scanning the oral cavity of a patient, which is an object, using the intraoral scanner.

In the present specification, an “object” may include teeth, gingiva, at least a partial region of the oral cavity, and/or artificial structures (e.g., orthodontic appliances, implants, artificial teeth, orthodontic aids inserted into the mouth, etc.) that may be inserted into the oral cavity. Here, the orthodontic appliance may include at least one of a bracket, an attachment, an orthodontic screw, a lingual orthodontic device, and a removable orthodontic retainer.

Hereinafter, embodiments will be described in detail with reference to the drawings.

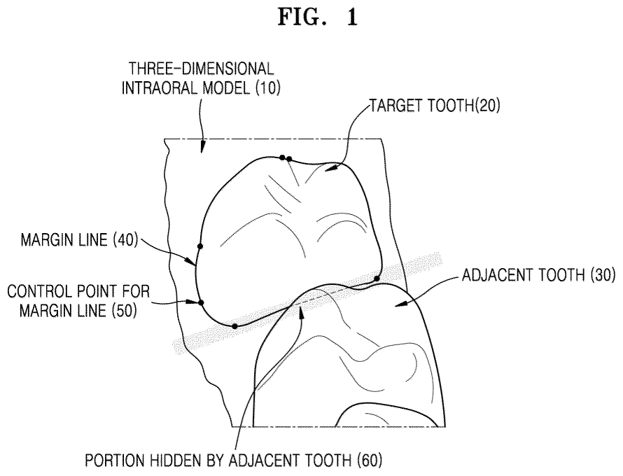

FIG. 1 is a reference diagram for explaining a method of setting a margin line in a three-dimensional intraoral model, according to related technology.

Referring to FIG. 1, a three-dimensional intraoral model 10 acquired by processing raw data obtained by scanning a patient's actual teeth or a plaster model created based on the patient's actual teeth may include one or more teeth.

One target tooth 20 on which a margin line 40 is to be set may be selected from among one or more teeth included in the three-dimensional intraoral model 10. For example, when the target tooth 20 is selected in the three-dimensional intraoral model 10, the margin line 40 may be set automatically by a program or manually by user selection. In this case, in either case, whether the margin line 40 is set automatically or manually, it is difficult to precisely set the margin line 40 in a portion 60 that is hidden by an adjacent tooth 30 adjacent to the target tooth 20. That is, for a portion between teeth, that is, a portion hidden by the adjacent tooth 30 based on the target tooth 20, a user may obtain an accurate margin line by more precisely modifying a control point 50 for the margin line. In this case, in order for a user to more easily control the control point 50 for the margin line for the portion hidden by the adjacent tooth 30, it is desirable to make it easier for the user to check the margin line of the target tooth 20 by processing and displaying the adjacent tooth 30, which partially hides the target tooth 20, to be transparent.

Therefore, in embodiments disclosed in the present disclosure, when a three-dimensional intraoral model is displayed, teeth that hide a target tooth are processed to be transparent and hidden, and displayed based on the target tooth, and thus, a user may set the margin line more precisely and easily by considering the degree of contact between the target tooth and adjacent teeth.

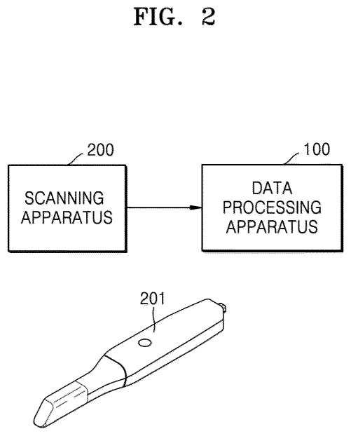

FIG. 2 is a diagram for explaining a digital intraoral model processing system according to an embodiment.

Referring to FIG. 2, the digital intraoral model processing system may include a scanning apparatus 200 and a data processing apparatus 100.

The scanning apparatus 200 scans an object, and the object may include any object or body to be scanned. For example, the object may include at least a part of a patient's body including an oral cavity or face, or a tooth model. The scanning apparatus 200 may include a handheld scanner for scanning an object while a user holds the handheld scanner, or a model scanner for installing a tooth model and scanning while moving around the installed tooth model.

For example, an intraoral scanner 201, which is a type of handheld scanner, may be a device for acquiring an image of an oral cavity including at least one tooth by being inserted into the oral cavity and scanning teeth in a non-contact manner. In addition, the intraoral scanner 201 may have a form that may be drawn in and out of the oral cavity, and scans the inside of the patient's oral cavity using at least one image sensor (e.g., an optical camera, etc.). The intraoral scanner 201 may acquire surface information on the object as raw data to image the surface of at least one of teeth, gingiva, and artificial structures (e.g., orthodontic devices including brackets and wires, implants, artificial teeth, orthodontic aids inserted into the oral cavity, etc.) insertable into the oral cavity, which are objects. The intraoral scanner 201 is suitable for scanning the oral cavity as it is in a form that is easy to enter and withdraw into the oral cavity. However, of course, body parts, such as the patient's face may also be scanned using the intraoral scanner 201.

The scanning apparatus 200 may acquire image data using an optical triangulation method, a confocal method, or other methods.

The image data acquired by the scanning apparatus 200 may be transmitted to the data processing apparatus 100 connected through a wired or wireless communication network.

The data processing apparatus 100 may be any electronic device capable of being connected to the scanning apparatus 200 through a wired or wireless communication network, receiving a two-dimensional image obtained by scanning an oral cavity from the scanning apparatus 200, and generating, processing, displaying, and/or transmitting an intraoral image based on the received two-dimensional image.

The data processing apparatus 100 may generate at least one of information generated by processing two-dimensional image data and an intraoral image generated by processing two-dimensional image data based on the two-dimensional image data received from the scanning apparatus 200, and display the generated information and the intraoral image through a display.

The data processing apparatus 100 may be a computing device such as a smart phone, a laptop computer, a desktop computer, a PDA, or a tablet PC, but is not limited thereto.

Also, the data processing apparatus 100 may exist in the form of a server (or server apparatus) for processing an intraoral image.

Also, the scanning apparatus 200 may transmit raw data obtained through scanning to the data processing apparatus 100 as it is. In this case, the data processing apparatus 100 may generate a three-dimensional intraoral image representing the oral cavity three-dimensionally based on the received raw data. In addition, the “three-dimensional intraoral image” may be generated by modeling the internal structure of the oral cavity based on the received raw data in three dimensions, and may be referred to as a “three-dimensional intraoral model”, a “digital intraoral model”, or a “three-dimensional intraoral image”. Hereinafter, a model or image representing the oral cavity in two or three dimensions is collectively referred to as a “three-dimensional intraoral model”.

Also, the data processing apparatus 100 may analyze, process, display, and/or transmit the generated intraoral image to an external device.

As another example, the scanning apparatus 200 may acquire raw data through a scan, process the obtained raw data to generate an image corresponding to the oral cavity, which is an object, and transmit the image to the data processing apparatus 100. In this case, the data processing apparatus 100 may analyze, process, display, and/or transmit the received image.

In the disclosed embodiment, the data processing apparatus 100 is an electronic device capable of generating and displaying an intraoral image representing an oral cavity including one or more teeth in three dimensions, which will be described in detail below.

According to an embodiment, when the data processing apparatus 100 receives raw data obtained by scanning an oral cavity from the scanning apparatus 200, the data processing apparatus 100 may process the received raw data to generate a three-dimensional intraoral model.

According to an embodiment, the data processing apparatus 100 may display an intraoral model including one or more teeth.

According to an embodiment, the data processing apparatus 100 may select a target tooth on which a margin line is to be set from among the one or more teeth included in the intraoral model and may display the margin line on the selected target tooth.

According to an embodiment, the data processing apparatus 100 may generate a clipping plane at a predetermined distance from the target tooth in a screen direction and may process and display to be transparent the area of the intraoral model included in a transparent processing area located in the screen direction based on the clipping plane.

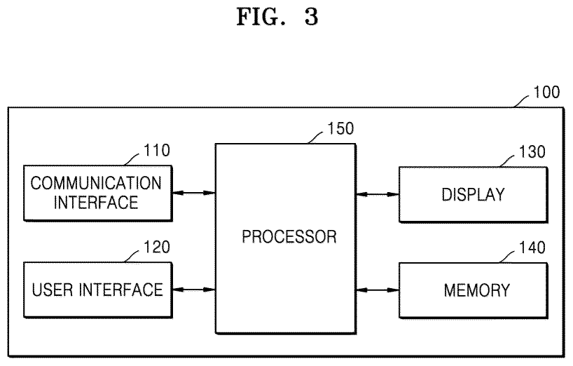

FIG. 3 is a block diagram illustrating a data processing apparatus 100 according to an embodiment.

Referring to FIG. 3, the data processing apparatus 100 may include a communication interface 110, a user interface 120, a display 130, a memory 140, and a processor 150.

The communication interface 110 may communicate with at least one external electronic device through a wired or wireless communication network. In particular, the communication interface 110 may communicate with the scanning apparatus 200 under the control of the processor 150. The communication interface 110 may communicate with an external electronic device or server connected through a wired/wireless communication network under the control of the processor.

The communication interface 110 may communicate with an external electronic device (e.g., an intraoral scanner, a server, or an external medical device) through a wired or wireless communication network. In particular, the communication interface may include at least one short-distance communication module for performing communication according to communication standards such as Bluetooth, Wi-Fi, Bluetooth Low Energy (BLE), NFC/RFID, Wifi Direct, UWB, or ZIGBEE.

In addition, the communication interface 110 may further include a long-distance communication module that communicates with a server for supporting long-distance communication according to the long-distance communication standard. In particular, the communication interface 110 may include a long-distance communication module for performing communication through a network for Internet communication. In addition, the communication interface may include a long-distance communication module for performing communication through a communication network conforming to a communication standard such as 3G, 4G, and/or 5G.

In addition, the communication interface 110 may include at least one port for connecting to an external electronic device by a wired cable to communicate with an external electronic device (e.g., intraoral scanner, etc.) by wire. Accordingly, the communication interface 110 may communicate with an external electronic device connected by wire through at least one port.

The user interface 120 may receive a user input for controlling the data processing apparatus. The user interface 120 may include a user input device including a touch panel for sensing a user's touch, a button for receiving a user's push operation, a mouse or keyboard for designating or selecting a point on the user interface screen, and the like, but is not limited thereto.

Also, the user interface 120 may include a voice recognition device for voice recognition. For example, the voice recognition device may be a microphone, and the voice recognition device may receive a user's voice command or voice request. Accordingly, the processor may control an operation corresponding to a voice command or a voice request to be performed.

The display 130 displays a screen. In particular, the display 130 may display a preset screen under the control of the processor 150. In particular, the display 130 may display a user interface screen including an intraoral image generated based on data obtained by scanning the oral cavity of a patient in the scanning apparatus 200. Alternatively, the display 130 may display a user interface screen including information related to a patient's dental treatment.

The memory 140 may store at least one instruction. Also, the memory 140 may store at least one instruction to be executed by the processor. Also, the memory may store at least one program executed by the processor 150. In addition, the memory 140 may store data received from the intraoral scanner (e.g., raw data obtained through intraoral scan, etc.). Alternatively, the memory may store an intraoral image representing the oral cavity in three dimensions.

The processor 150 performs at least one instruction stored in the memory 140 to control an intended operation to be performed. Here, at least one instruction may be stored in an internal memory included in the processor 150 or a memory 140 included in the data processing apparatus separately from the processor.

In particular, the processor 150 may perform at least one instruction to control at least one configuration included in the data processing apparatus so that an intended operation is performed. Therefore, even if the processor performs certain operations as an example, the processor may control at least one component included in the data processing apparatus so that preset operations are performed.

According to an embodiment, by executing one or more instructions stored in the memory 140, the processor 150 may display an intraoral model including one or more teeth, select a target tooth from among the one or more teeth included in the intraoral model, display a margin line on the selected target tooth, generate a clipping plane at a location spaced apart from the target tooth by a predetermined distance in a screen direction, and process and display to be transparent the area of the intraoral model included in a transparent processing area located in the screen direction based on the clipping plane.

According to an embodiment, by executing one or more instructions stored in the memory 140, the processor 150 may provide the margin line displayed on the target tooth such that the position of the margin line is adjustable, and adjust and display the position of the margin line according to a user input for adjusting the position of the margin line.

According to an embodiment, by executing one or more instructions stored in the memory 140, the processor 150 may provide one or more control points to the margin line and adjust the position of the margin line according to a user input for moving the positions of the one or more control points. The predetermined distance may include a predetermined distance from the center point of a bounding box surrounding the target tooth. The predetermined distance may include an average value of the horizontal and vertical lengths of the xz plane of the bounding box or a value obtained by adding a predetermined offset to the median value of the distance from the center point of the target tooth to the center point of an adjacent tooth.

According to an embodiment, by executing one or more instructions stored in the memory 140, the processor may process and display to be transparent the area of the intraoral model included in the transparent processing area when the size of the target tooth displayed on a display is greater than or equal to a threshold, and may display the intraoral model without setting a transparent processing area when the size of the target tooth displayed on the display is less than the threshold.

According to an embodiment, by executing one or more instructions stored in the memory 140, the processor 150 may display a predetermined icon and remove the intraoral image area that is processed to be transparent, by removing the clipping plane according to an input for selecting the icon.

According to an embodiment, by executing one or more instructions stored in the memory 140, the processor 150 may rotate and display the intraoral model in response to a user input for rotating the intraoral model, generate a new clipping plane at the predetermined distance in the screen direction from the target tooth rotated according to the rotation of the intraoral model and generate a new transparent processing area based on the new clipping plane, and process and display to be transparent the area of the intraoral model included in the new transparent processing area.

According to an embodiment, by executing one or more instructions stored in the memory 140, the processor 150 may detect whether the angle between the screen direction and an occlusal direction of the target tooth, which has been rotated in response to a user input for rotating the intraoral model, is less than a critical angle. In addition, when the angle is detected to be less than the critical angle, the processor may remove a portion processed to be transparent by the transparent processing area by removing the clipping plane.

According to an embodiment, by executing one or more instructions stored in the memory 140, the processor 150 may exclude at least a portion of the target tooth from a transparent processing display when the at least a portion of the target tooth is located in the transparent processing area according to the rotational movement of the target tooth in response to a user input for rotating the intraoral model.

According to an example, the processor 150 may be implemented in a form that internally includes at least one internal processor and a memory device (e.g., RAM, ROM, etc.) for storing at least one of programs, instructions, signals, and data to be processed or used by the internal processor.

In addition, the processor 150 may include a graphic processing unit for processing a graphic corresponding to a video. In addition, the processor 150 may be implemented as a system on chip (SoC) in which a core and a GPU are integrated. Also, the processor 150 may include a single core or multiple cores. For example, the processor may include a dual-core, triple-core, quad-core, hexa-core, octa-core, deca-core, dodeca-core, hexa-dash-vale core, and the like.

In the disclosed embodiment, the processor 150 may generate an intraoral image based on a two-dimensional image received from the scanning apparatus 200.

In particular, under the control of the processor 150, the communication interface 110 may receive data obtained from the scanning apparatus 200, for example, raw data obtained through an intraoral scan. In addition, the processor 150 may generate a three-dimensional intraoral image representing the oral cavity three-dimensionally based on the raw data received from the communication interface. For example, to reconstruct a three-dimensional image according to the optical triangulation method, and the intraoral scanner may include at least one camera and, in a specific embodiment, may include an L camera corresponding to a left field of view and an R camera corresponding to a right field of view. In addition, the intraoral scanner may acquire L image data corresponding to the left field of view and R image data corresponding to the right field of view from the L camera and the R camera, respectively. Subsequently, the intraoral scanner (not shown) may transmit raw data including L image data and R image data to the communication interface of the data processing apparatus 100.

Then, the communication interface 110 may transmit the received raw data to the processor, and the processor may generate an intraoral image representing the oral cavity in three dimensions based on the received raw data.

In addition, the processor 150 may directly receive an intraoral image representing the oral cavity from an external server, a medical device, or the like by controlling the communication interface. In this case, the processor may acquire a three-dimensional intraoral image without generating a three-dimensional intraoral image based on the raw data.

According to the disclosed embodiment, that the processor 150 performs operations such as ‘extract’, ‘acquire’, and ‘generate’ may include controlling other components to perform the above-described operations in addition to directly performing the above-described operations by executing at least one instruction in the processor 150.

To implement the embodiments disclosed in the present disclosure, the data processing apparatus 100 may include only some of the components illustrated in FIG. 3, or may include more components in addition to the components illustrated in FIG. 3.

In addition, the data processing apparatus 100 may store and execute dedicated software linked to the intraoral scanner. Here, the dedicated software may be called a dedicated program, a dedicated tool, or a dedicated application. When the data processing apparatus 100 operates in conjunction with the scanning apparatus 200, dedicated software stored in the data processing apparatus 100 may be connected to the scanning apparatus 200 to receive data acquired through an intraoral scan in real time. For example, there is dedicated software for processing data acquired through intraoral scans in Medit's intraoral scanner. In particular, Medit produces and distributes dedicated software for processing, managing, using, and/or transmitting data acquired from intraoral scanners. Here, “dedicated software” refers to a program, tool, or application that may be operated in conjunction with the intraoral scanner, such that “dedicated software” may be commonly used by various intraoral scanners developed and sold by various manufacturers. In addition, the dedicated software described above may be produced and distributed separately from the intraoral scanner that performs the intraoral scan.

The data processing apparatus 100 may store and execute dedicated software corresponding to an intraoral scanner product. The dedicated software may perform at least one operation to acquire, process, store, and/or transmit the intraoral image. Here, the dedicated software may be stored in the processor. In addition, dedicated software may provide a user interface for use of data acquired from the intraoral scanner. Here, the user interface screen provided by the dedicated software may include an intraoral image generated according to the disclosed embodiment.

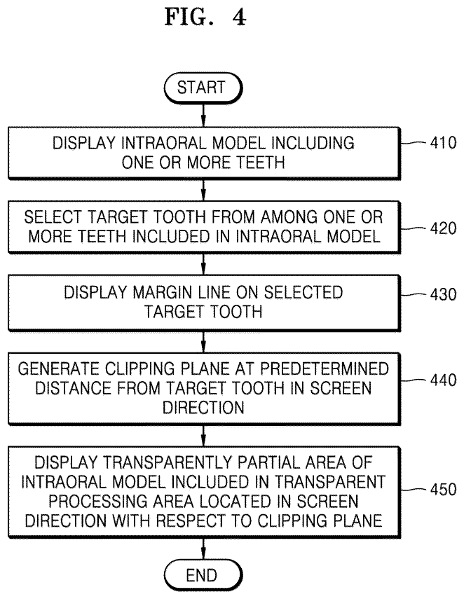

FIG. 4 is a flowchart illustrating a method of processing a three-dimensional intraoral model in a data processing apparatus according to an embodiment. The three-dimensional intraoral model processing method illustrated in FIG. 4 may be performed through the data processing apparatus 100. Accordingly, the three-dimensional intraoral model processing method illustrated in FIG. 4 may be a flowchart illustrating operations of the data processing apparatus 100.

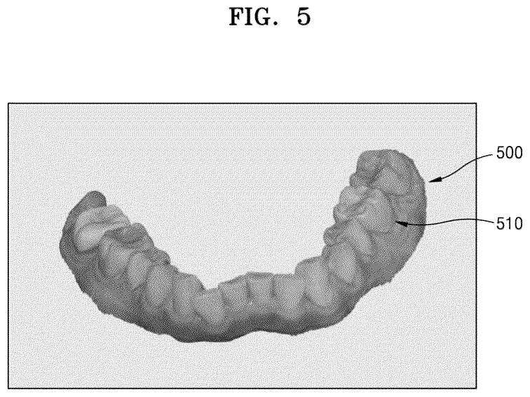

Referring to FIG. 4, in operation 410, the data processing apparatus 100 may acquire a three-dimensional intraoral model 500 (see FIG. 5).

The data processing apparatus 100 may receive, from the scanning apparatus 200, data obtained by scanning an intraoral in a patient or by scanning a tooth model, and acquire and display a three-dimensional intraoral model including one or more teeth by processing the received data. As another example, the data processing apparatus 100 may import a file containing data obtained by scanning an intraoral or scanning a tooth model from an external source.

FIG. 5 illustrates an example of a three-dimensional intraoral model obtained by the data processing apparatus 100 according to an embodiment.

For example, when two-dimensional data is obtained using an intraoral scanner, the data processing apparatus 100 may calculate coordinates of a plurality of illuminated surface points using a triangulation method. As the amount of scan data increases by scanning the surface of the object while moving using the intraoral scanner, coordinates of the surface points may be accumulated. As a result of this image acquisition, a point cloud of vertices may be identified to represent the extent of the surface. Points in the point cloud may represent actual measured points on the three-dimensional surface of the object. The surface structure may be approximated by forming a polygonal mesh in which adjacent vertices of a point cloud are connected by line segments. The polygonal mesh may be variously determined, such as a triangular, quadrangular, or pentagonal mesh. The relationship between the polygons of the mesh model and the neighboring polygons may be used to extract features of the tooth boundary, for example, curvature, smallest curvature, edge, spatial relationship, and the like.

Returning to FIG. 4, in operation 420, the data processing apparatus 100 may select a target tooth from among one or more teeth included in the three-dimensional intraoral model.

According to an embodiment, the data processing apparatus 100 may select a target tooth from among one or more teeth included in the three-dimensional intraoral model according to a user input for selecting the target tooth. The data processing apparatus 100 may select a target tooth by identifying the boundary of the target tooth selected by a user by using characteristics of the tooth boundary, such as curvature, minimum curvature, edge, and spatial relationship. For example, it is shown that a target tooth 510 is selected from a three-dimensional intraoral model 500 shown in FIG. 5.

According to an embodiment, the data processing apparatus 100 may automatically select a target tooth from among one or more teeth included in the three-dimensional intraoral model according to a predetermined algorithm.

Returning to FIG. 4, in operation 430, the data processing apparatus 100 may display a margin line on the selected target tooth.

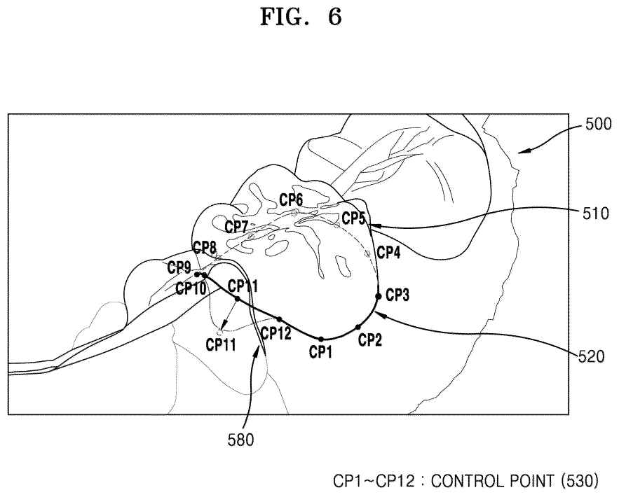

FIG. 6 is a diagram illustrating a margin line displayed on a target tooth, according to an example.

Referring to FIG. 6, the data processing apparatus 100 may display a margin line 520 on the boundary between the target tooth 510 and the gingiva selected from the three-dimensional intraoral model 500.

In addition, the data processing apparatus 100 may arrange one or more control points 530 to adjust the margin line 520. In FIG. 6, it is shown that the control points 530 include a control point CP1 to a control point CP12. When one of the control points 530 is selected from a user and an input for moving the position of the selected control point 530 is received, the data processing apparatus 100 may adjust the margin line 520 in response to the position movement of the control point 530. For example, when the control point CP11 is selected from a user and an input is received to drag the selected control point CP11 and move it to the position CP11′, the data processing apparatus 100 may adjust the margin line 510 to a new margin line consisting of the control points CP1, . . . , CP10, CP11′, and CP12.

Referring to FIG. 6, at the control points CP9, CP10, and CP11 arranged at an area in which the target tooth 510 and the adjacent tooth 580 are adjacent to each other, the boundary between the target tooth 510 and the gingiva may not be identified due to the adjacent tooth 580, making it difficult for a user to adjust control points. Therefore, according to the embodiments disclosed in the present disclosure, the data processing apparatus 100 may process a tooth portion adjacent to the target tooth 510, to be transparent, thereby allowing the user to easily set the margin line of the target tooth 510.

Returning to FIG. 4, in operation 440, the data processing apparatus 100 may generate a clipping plane at a predetermined distance from the target tooth in the screen direction. The clipping plane may represent a reference plane for making a partial area of the three-dimensional intraoral model transparent. The clipping plane may be understood as a plane that is parallel to the screen of the data processing apparatus 100 and has a depth greater than the depth of the screen.

In operation 450, the data processing apparatus 100 may process and display to be transparent a partial area of the intraoral model included in the transparent processing area located in the screen direction with respect to the clipping plane.

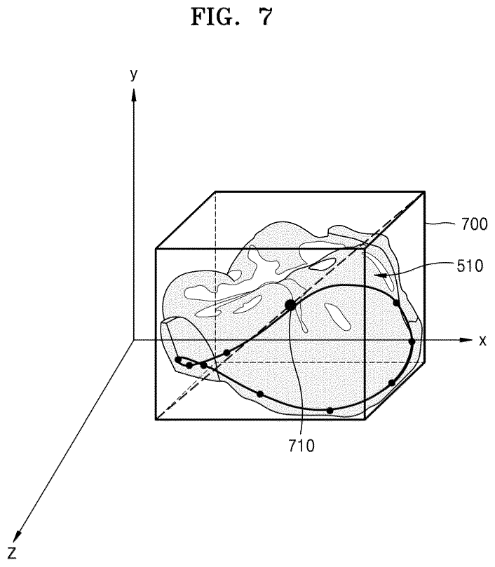

Now, a method of generating a clipping plane and processing and displaying to be transparent a partial area of an intraoral model is described in detail with reference to FIGS. 7 to 12.

FIG. 7 is a reference diagram for explaining a method of finding the center point of a target tooth, according to an embodiment.

Referring to FIG. 7, the data processing apparatus 100 may generate a bounding box 700 based on a target tooth 510. The bounding box 700 has a rectangular parallelepiped shape connecting the minimum point of the target tooth 510 to the maximum point of the target tooth 510, and the diagonal center of the rectangular parallelepiped shape may be determined as a center point 710 of the target tooth 510. There is one center point for each tooth, and the center point does not change even when the screen is rotated. Even when a user rotates the three-dimensional intraoral model displayed on the display, the screen may be rotated around the center point 710 of the target tooth 500.

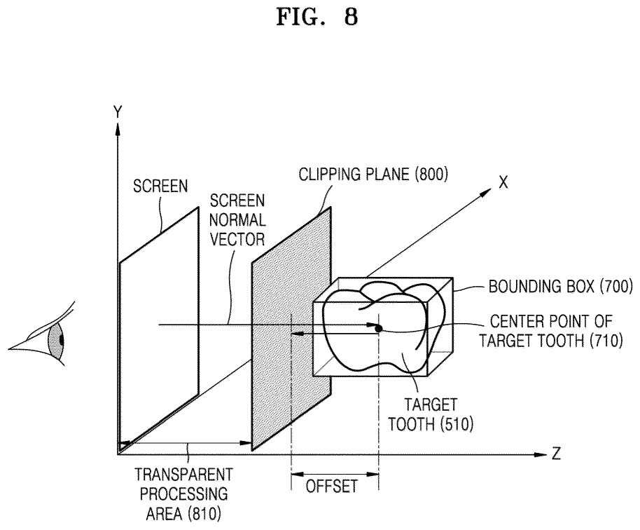

FIG. 8 is a reference diagram for explaining an example of a method of generating a clipping plane, according to an embodiment.

Referring to FIG. 8, the data processing apparatus 100 may find a screen normal vector that passes through a screen in a vertical direction, offset a certain distance from a center point 710 of a target tooth 510 in an opposite direction of the screen normal vector, that is, in the screen direction, and generate a clipping plane 800 in the vertical direction at an offset position. In addition, the data processing apparatus 100 may set, as a transparent processing area, an area in the screen direction based on the clipping plane 800. The data processing apparatus 100 may process and display to be transparent a partial area of the three-dimensional intraoral model that is located in the transparent processing area. In this way, by setting the transparent processing area at a predetermined distance based on the center point of the target tooth, no matter which direction a user moves or rotates the three-dimensional intraoral model, a portion of the three-dimensional intraoral model included in the transparent processing area is always processed to be transparent at the predetermined distance based on the center point of the target tooth, and thus, a boundary portion of the target tooth may be easily identified.

In this way, because the clipping plane 800 is generated at a position offset by a certain distance from the center point of the target tooth for which a margin line is to be set, a location where the clipping plane is generated varies depending on the position movement of the target tooth. A user may move the position of the three-dimensional intraoral model including the target tooth to set the margin line of the target tooth or for various other reasons. Here, the movement may include any positional movement, such as rotating, vertically moving, or horizontally moving the three-dimensional intraoral model. Therefore, the position of the center point of the target tooth may continuously change according to a user's movement operation of the three-dimensional intraoral model. In this way, even when the center point of the target tooth continues to change, the clipping plane is generated at a position offset by a predetermined distance from the center point of the target tooth, and thus, the clipping plane may always be generated at a position at a certain distance from the center point of the target tooth. In addition, because the data processing apparatus 100 sets the transparent processing area based on the clipping plane generated in this way, the transparent processing area may ultimately be set at a position at a certain distance from the center point of the target tooth. For example, as the data processing apparatus 100 sets a certain distance from the center point of the target tooth as the position where an adjacent tooth starts, no matter which direction or how much a user moves the three-dimensional intraoral model, the transparent processing area may always be set and displayed from the position where the adjacent tooth starts. Therefore, when a user sets or modifies the margin line of the target tooth, the adjacent tooth may be displayed to be transparent, making it possible to more easily identify a boundary portion of the target tooth.

Here, the data processing apparatus 100 may determine, according to various methods, a predetermined distance offset from the center point 710 of the target tooth in order to generate the clipping plane.

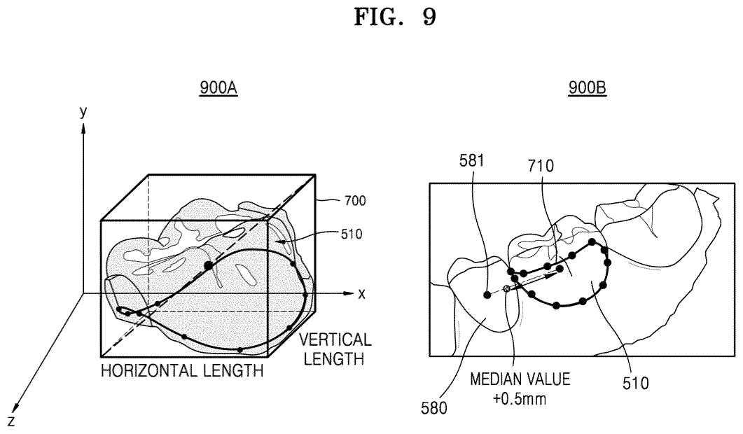

FIG. 9 is a reference diagram for explaining a method of determining a predetermined distance offset from a center point 710 of a target tooth to generate a clipping plane, according to an embodiment.

Referring to 900A of FIG. 9, the data processing apparatus 100 may determine the predetermined distance as the average value of the horizontal and vertical lengths in a plane perpendicular to an occlusal direction in a bounding box of the target tooth. Here, the reason for determining the predetermined distance based on the length on the plane perpendicular to the occlusal direction is because a clipping plane is generated as a reference for displaying an adjacent tooth portion to be transparent in order to set or modify the margin line of the target tooth and thus the occlusal direction of the target tooth and the distance (distance in the y-axis in FIG. 9) in the occlusal direction are mostly meaningless. In FIG. 9, because the occlusal direction is in the y-axis direction, the plane perpendicular to the occlusal direction may be the xz plane. However, the occlusal direction may be the x-axis direction or the z-axis direction depending on embodiments.

However, according to another embodiment, the data processing device 100 may determine the predetermined distance as an average value of the horizontal length, vertical length, and height by considering all distances on the xyz axis of the bounding box of the target tooth.

Referring to 900B of FIG. 9, the data processing apparatus 100 may determine the predetermined distance based on the distance between the center point of the target tooth and the center point of the adjacent tooth. Specifically, the data processing apparatus 100 may calculate the median value of the distance between the center point 710 of the target tooth 510 and the center point 581 of the adjacent tooth 580, and may determine the predetermined distance as a value obtained by adding a predetermined value (for example, +0.5 mm) to the obtained median value.

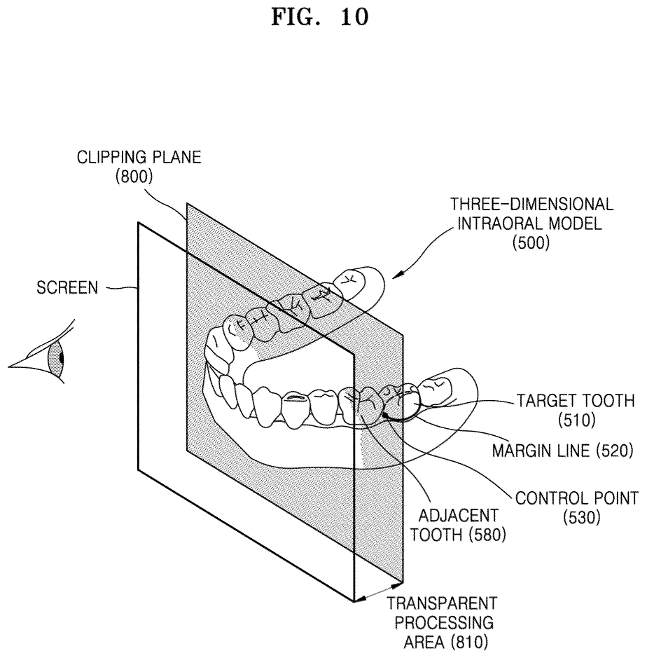

FIG. 10 is a diagram showing that an intraoral model area located in a transparent processing area set based on a clipping plane is displayed to be transparent, according to an embodiment.

Referring to FIG. 10, the data processing apparatus 100 may process and display to be transparent a partial area 1000 of a three-dimensional intraoral model 500 included in a transparent processing area 810 in a screen direction with respect to a clipping plane 800. In this way, as an area at a predetermined distance based on the target tooth 510, that is, an intraoral model area 1000 included in the transparent processing area 810, is processed to be transparent to be transparent so as to hide the adjacent tooth 580, the user may more easily identify a boundary area of the target tooth 510. Accordingly, the user may more precisely move and position the control point 530 for setting the margin line 520 of the target tooth 510, thereby enabling the user to obtain the margin line 520 more accurately.

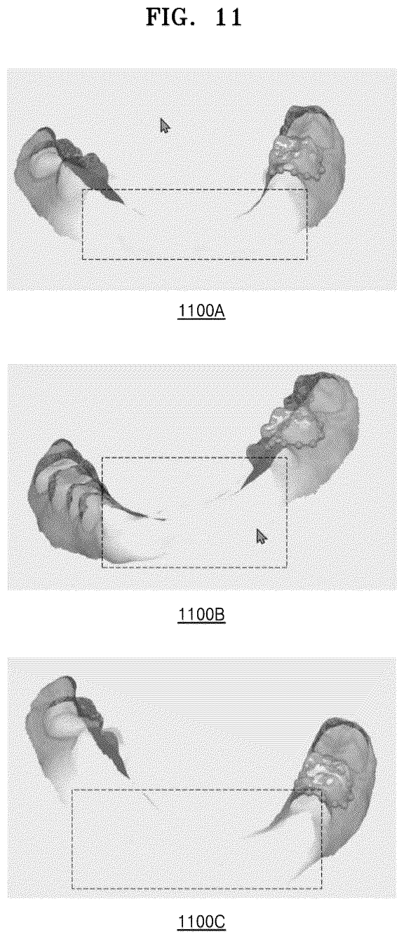

FIG. 11 is a diagram illustrating an example in which an area displayed to be transparent changes as the position of a three-dimensional intraoral model moves, according to an embodiment.

Referring to 1100A, 1100B, and 1100C of FIG. 11, a user may display a three-dimensional intraoral model from various angles by moving the three-dimensional intraoral model in parallel, vertically, or rotationally to set a margin line of a target tooth in the three-dimensional intraoral model shown on a display. No matter what angle the user rotates the three-dimensional intraoral model, the clipping plane according to the disclosed embodiment may be generated at a position offset by a predetermined distance around the target tooth, for example, at a position where the adjacent tooth starts, and may process and display to be transparent an intraoral model area included in the transparent processing area, based on the position, and thus, the user may easily identify the boundary of the target tooth for which a margin line is to be set.

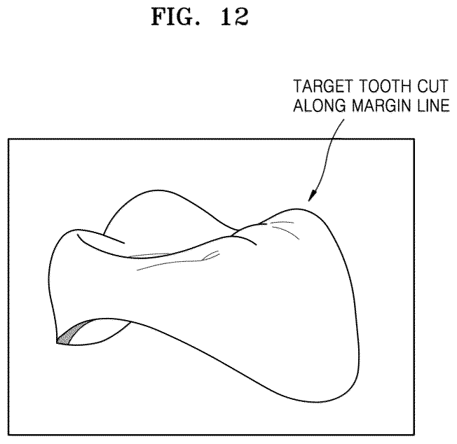

When the user's margin line setting is completed, the data processing apparatus 100 may display the target tooth cut along the margin line. FIG. 12 shows a target tooth cut along a margin line, according to an example.

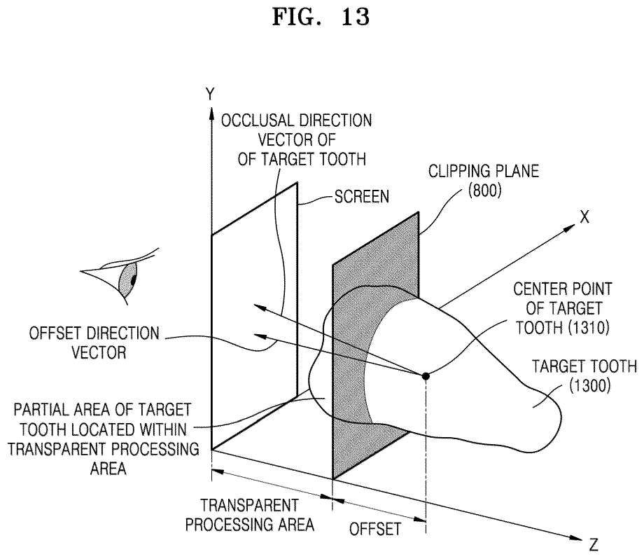

FIG. 13 is a reference diagram for explaining a processing method when a portion of a target tooth is included in a transparent processing area, according to an embodiment.

When a target tooth 1300 is long in an occlusal direction, a portion of the target tooth 1300 may be included in the transparent processing area while a user rotates a three-dimensional intraoral model.

Referring to FIG. 13, a clipping plane may be generated at a position corresponding to an offset determined by considering only the horizontal and vertical lengths of the target tooth 1300 on the xz plane without considering the length of the target tooth 1300 in the occlusal direction. The user may rotate the three-dimensional intraoral model to various angles. When the angle between an occlusal direction vector of the target tooth 1300, which is long in the occlusal direction, and a vector (hereinafter, referred to as an offset direction vector) in an opposite direction to a screen normal vector is less than a predetermined angle, that is, when an occlusal surface of the target tooth 1300 faces toward the screen, a portion of the target tooth 1300 may be included in the transparent processing area. Therefore, even though it is the target tooth 1300, a partial area of the target tooth 1300 located within the transparent processing area may be processed to be transparent and hidden. However, in this case, contrary to the purpose of enabling easy identification of the boundary area of the target tooth, a portion of the target tooth itself may not be displayed, resulting in an awkward display.

Therefore, in such a situation, according to an embodiment, the data processing apparatus 100 may prevent the transparent processing area from occurring by not generating a clipping plane and display the entire three-dimensional intraoral model on the display.

Alternatively, according to another embodiment, the data processing apparatus 100 may control the portion of the target tooth 1300 included in the transparent processing area to be displayed as is without any hiding process. In other words, when there is a part corresponding to the target tooth 1300 in the area of the three-dimensional intraoral model included in the transparent processing area, the data processing apparatus 100 may control the part corresponding to the target tooth 1300 to be displayed as is without making it being transparent.

According to an embodiment, the data processing apparatus 100 may be controlled to toggle between displaying and not displaying the transparent processing area according to a simple user input.

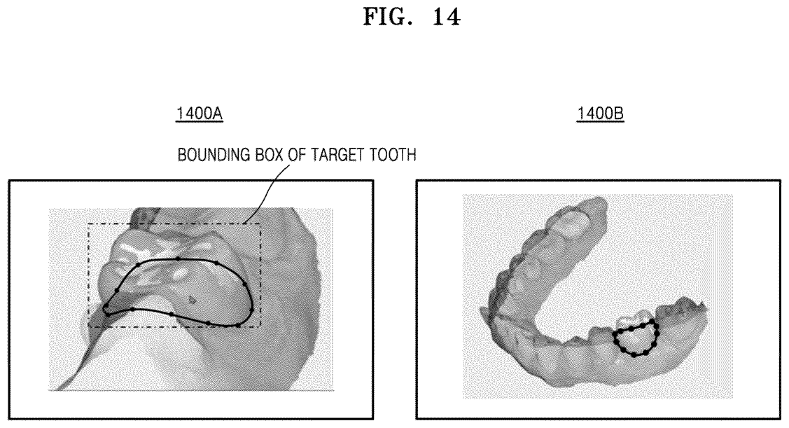

FIG. 14 is a reference diagram for explaining how a transparent processing area is toggled between a displayed state and a non-displayed state according to a user input, according to an embodiment.

The data processing apparatus 100 processes and displays to be transparent an area around the target tooth so that a user may accurately set the margin line of the target tooth, but the user may also wish to view a three-dimensional intraoral model, which is not processed to be transparent, as a whole. Accordingly, when receiving a predetermined user input, the data processing apparatus 100 may process the three-dimensional intraoral model so that the three-dimensional intraoral model may be displayed without being hidden by using a clipping plane.

Referring to 1400A of FIG. 14, according to a user input that controls the bounding box of the target tooth 510 of the three-dimensional intraoral model to zoom fit to the entire screen of the display, the data processing apparatus 100 may generate a clipping plane according to the embodiments disclosed in the present disclosure, set a transparent processing area based on the clipping plane, and process and display to be transparent a portion of the three-dimensional intraoral model area located in the transparent processing area. The fact that a user zooms in to enlarge the target tooth to fill the entire screen may be interpreted as the user's intention to accurately set the margin line of the target tooth, and thus, in this case, the data processing apparatus 100 may facilitate the user's setting of the margin line by processing an area adjacent to the target tooth to be transparent.

Referring to 1400B of FIG. 14, the fact that a user zooms out the three-dimensional intraoral model to display the entire three-dimensional intraoral model on the screen may be understood as the user wishing to view the three-dimensional intraoral model as a whole rather than viewing the margin line of a specific target tooth. Therefore, when the three-dimensional intraoral model is displayed as a whole on the display screen by a zoom-out input from the user, the data processing apparatus 100 may display the entire three-dimensional intraoral model without any portion being transparent by the transparent processing area.

When a three-dimensional intraoral model is displayed, the criteria for determining whether to hide a portion of the three-dimensional intraoral model by a transparent processing area may be determined in various ways.

According to an embodiment, as shown in FIG. 14, according to a user input that causes the bounding box of the target tooth to be fully displayed on the display screen and other teeth to be only partially displayed on the display screen, the data processing apparatus 100 may set a transparent processing area and process and display to be transparent a partial area of a three-dimensional intraoral model. In other cases, that is, according to a user input that causes the shapes of one or more other teeth to be completely displayed including the bounding box of the target tooth on the display screen, the data processing apparatus 100 may display a three-dimensional intraoral model without setting a transparent processing area.

According to an embodiment, when the size of the bounding box of the target tooth 510 exceeds a threshold according to a user input, the data processing apparatus 100 may set a transparent processing area and process and display to be transparent a partial area of the three-dimensional intraoral mode, and when the size of the bounding box does not exceed the threshold, the data processing apparatus 100 may display the three-dimensional intraoral model without setting a transparent processing area.

According to an embodiment, the data processing apparatus 100 may provide a pre-designated icon in a partial area of the display and may set a transparent processing area according to a user input for selecting the pre-designated icon and process and display to be transparent a partial area of a three-dimensional intraoral model or display the three-dimensional intraoral model without setting a transparent processing area.

The intraoral image processing method according to an embodiment of the present disclosure may be implemented in the form of program instructions that may be executed through various computer means and recorded in a computer-readable medium. Also, an embodiment of the present disclosure may be a computer-readable storage medium in which one or more programs including at least one instruction for executing an intraoral image processing method are recorded.

The computer-readable storage medium may include program instructions, data files, data structures, etc. alone or in combination. Here, examples of computer-readable storage media may include hardware devices configured to store and execute program instructions, for example, magnetic media such as hard disks, floppy disks and magnetic tapes, optical media such as CD-ROM and DVD, magneto-optical media such as a floptical disk, and ROM, RAM, flash memory, etc.

Here, the device-readable storage medium may be provided in the form of a non-transitory storage medium. Here, the ‘non-transitory storage medium’ may mean that the storage medium is a tangible device. Also, the ‘non-transitory storage medium’ may include a buffer in which data is temporarily stored.

According to one embodiment, the intraoral image processing method according to various embodiments disclosed herein may be included in a computer program product and provided. The computer program product may be distributed in the form of a machine-readable storage medium (e.g., compact disc read only memory (CD-ROM)). Alternatively, the computer program produce may be distributed online (e.g., downloaded or uploaded) through an application store (e.g., play store, etc.) or directly between two user devices (e.g., smartphones). In particular, the computer program product according to the disclosed embodiment may include a storage medium in which a program including at least one instruction to perform the intraoral image processing method according to the disclosed embodiment is recorded.

Although embodiments have been described above in detail, the scope of the present disclosure is not limited thereto, and various modifications and alterations by those skill in the art using the basic concept of the present disclosure defined in the following claims also fall within the scope of the present disclosure.

1. A data processing apparatus comprising:
a memory storing one or more instructions; and
a processor configured to execute the one or more instructions to:
display an intraoral model including one or more teeth;
select a target tooth from among the one or more teeth included in the intraoral model; and
display transparently an area of the intraoral model included in a transparent processing area located in a screen direction based on a clipping plane located at a predetermined distance from the target tooth in the screen direction.

2. The data processing apparatus of claim 1, wherein the processor is further configured to, by executing the one or more instructions:
provide a margin line displayed on the target tooth such that a position of the margin line is adjustable; and
adjust and display the position of the margin line according to a user input for adjusting the position of the margin line.

3. The data processing apparatus of claim 1, wherein the predetermined distance includes a distance from a center point of a bounding box surrounding the target tooth.

4. The data processing apparatus of claim 3, wherein the predetermined distance includes an average value of horizontal and vertical lengths of a plane perpendicular to an occlusal direction in the bounding box or a value obtained by adding a predetermined offset to a median value of a distance from a center point of the target tooth to a center point of an adjacent tooth.

5. The data processing apparatus of claim 1, wherein the processor is further configured to, by executing the one or more instructions:
display transparently an area of the intraoral model included in the transparent processing area when a size of the target tooth displayed on a display is greater than or equal to a threshold; and
display the intraoral model without setting the transparent processing area when the size of the target tooth displayed on the display is less than the threshold.

6. The data processing apparatus of claim 1, wherein the processor is further configured to, by executing the one or more instructions:
display a predetermined icon; and
display the intraoral model without setting the transparent processing area by removing the clipping plane according to an input for selecting the icon.

7. The data processing apparatus of claim 1, wherein the processor is further configured to, by executing the one or more instructions:
rotate and display the intraoral model in response to a user input for rotating the intraoral model;
generate a new transparent processing area based on a new clipping plane by generating a new clipping plane at the predetermined distance in the screen direction from the target tooth rotated according to the rotation of the intraoral model; and
display transparently the area of the intraoral model included in the new transparent processing area.

8. The data processing apparatus of claim 1, wherein the processor is further configured to, by executing the one or more instructions:
detect whether an angle formed between an occlusal direction vector of the target tooth rotated in response to a user input for rotating the intraoral model, and a vector in an opposite direction to a screen normal vector is less than a threshold angle; and
remove the area processed to be transparent by the transparent processing area by removing the clipping plane based on the angle being detected to be less than the threshold angle.

9. The data processing apparatus of claim 1, wherein the processor is further configured to, by executing the one or more instructions,
exclude at least a portion of the target tooth from displaying transparently when the at least a portion of the target tooth is located in the transparent processing area according to rotational movement of the target tooth in response to a user input for rotating the intraoral model.

10. A method of operating a data processing apparatus, the method comprising:
displaying an intraoral model including one or more teeth;
selecting a target tooth from among the one or more teeth included in the intraoral model; and
displaying transparently an area of the intraoral model included in a transparent processing area located in a screen direction based on a clipping plane located at a predetermined distance from the target tooth in the screen direction.

11. The method of claim 10, further comprising:
providing a margin line displayed on the target tooth such that a position of the margin line is adjustable, and
adjusting and displaying the position of the margin line according to a user input for adjusting the position of the margin line.

12. The method of claim 10, wherein the predetermined distance includes a predetermined distance from a center point of a bounding box surrounding the target tooth.

13. The method of claim 12, wherein the predetermined distance includes an average value of horizontal and vertical lengths of a plane perpendicular to an occlusal direction in the bounding box or a value obtained by adding a predetermined offset to a median value of a distance from a center point of the target tooth to a center point of an adjacent tooth.

14. The method of claim 10, further comprising:
displaying transparently the area of the intraoral model included in the transparent processing area when a size of the target tooth displayed on a display is greater than or equal to a threshold; and
displaying the intraoral model without setting the transparent processing area when the size of the target tooth displayed on the display is less than the threshold.

15. The method of claim 10, further comprising:
rotating and displaying the intraoral model in response to a user input for rotating the intraoral model;
generating a new transparent processing area based on the new clipping plane by generating a new clipping plane at the predetermined distance in the screen direction from the target tooth rotated according to the rotation of the intraoral model; and
displaying transparently the area of the intraoral model included in the new transparent processing area.

16. The method of claim 10, further comprising:
detecting whether an angle formed between an occlusal direction vector of the target tooth rotated in response to a user input for rotating the intraoral model, and a vector in an opposite direction to a screen normal vector is less than a threshold angle; and
removing a portion processed to be transparent by the transparent processing area by removing the clipping plane when the angle is detected to be less than the threshold angle.

17. The method of claim 10, further comprising excluding at least a portion of the target tooth from displaying transparently when the at least a portion of the target tooth is located in the transparent processing area according to rotational movement of the target tooth in response to a user input for rotating the intraoral model.

18. A computer-readable recording medium comprising one or more instructions for performing a method of processing an intraoral model, wherein the method of processing the intraoral model comprises:
displaying an intraoral model including one or more teeth;
selecting a target tooth from among the one or more teeth included in the intraoral model; and
displaying transparently the area of the intraoral model included in a transparent processing area located in a screen direction based on a clipping plane located at a predetermined distance from the target tooth in the screen direction.
